(19) United States
(12) Patent Application Publication
(10) Pub. No.: US 2024/0293085 A1
(43) Pub. Date: Sep. 5, 2024

(54) SYSTEM, DEVICE AND METHOD OF DYNAMIC GLUCOSE PROFILE RESPONSE TO PHYSIOLOGICAL PARAMETERS

(71) Applicant: Abbott Diabetes care Inc., Alameda, CA (US)

(72) Inventors: Gary Alan Hayter, Oakland, CA (US); Nathan Christopher Crouther, San Francisco, CA (US); Michael Love, Pleasanton, CA (US); Erwin Satrya Budiman, Fremont, CA (US); Daniel Milfred Bernstein, El Granada, CA (US)

(73) Assignee: Abbott Diabetes care Inc., Alameda, CA (US)

(21) Appl. No.: 18/656,715

(22) Filed: May 7, 2024

Related U.S. Application Data
(63) Continuation of application No. 18/150,996, filed on Jan. 6, 2023.
Application No. 18/150,996 is a continuation of application No. 15/742,502, filed on Jan. 6, 2018, now Pat. No. 11,553,883.
(60) Provisional application No. 62/307,346, filed on Mar. 11, 2016.
(60) Provisional application No. 62/191,218, filed on Jul. 10, 2015.

(51) Int. Cl. A61B 5/00; A61B 5/024; A61B 5/11; A61B 5/145; A61B 5/1486; G16B 40/10; G16H 40/63

(52) U.S. Cl. CPC A61B 5/7246; A61B 5/0022; A61B 5/1118; A61B 5/1451; A61B 5/14532; A61B 5/4839; A61B 5/4866; A61B 5/7275; G16B 40/10; G16H 40/63; A61B 5/024; A61B 5/1114; A61B 5/1123; A61B 5/1486; A61B 2560/0223

(57) ABSTRACT

Method, device and system for providing consistent and reliable glucose response information to physiological changes and/or activities is provided to improve glycemic control and health management.

### RELATED APPLICATIONS

This application is a continuation of U.S. patent application Ser. No. 18/150,996 filed on Jan. 6, 2023, which is a continuation of U.S. patent application Ser. No. 15/742,502 filed on Jan. 6, 2018, now U.S. Pat. No. 11,553,883, which is a national stage patent application under 35 U.S.C. § 371 claims priority to PCT Application No. PCT/US16/41632 filed Jul. 8, 2016, which is related to U.S. Provisional Application No. 62/307,346 filed Mar. 11, 2016, U.S. Provisional Application No. 62/191,218 filed Jul. 10, 2015, and to U.S. Provisional Application No. 62/307,344 filed Mar. 11, 2016, entitled “Systems, Devices, and Methods For Meal information Collection, Meal Assessment, and Analyte Data Correlation,” the disclosures of each of which are incorporated herein by reference for all purposes.

### INCORPORATION BY REFERENCE

Patents, applications and/or publications described herein, including the following patents, applications and/or publications are incorporated herein by reference for all purposes: U.S. Pat. Nos. 4,545,382; 4,711,245; 5,262,035; 5,262,305; 5,264,104; 5,320,715; 5,356,786; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; 6,749,740; 6,764,581; 6,773,671; 6,881,551; 6,893,545; 6,932,892; 6,932,894; 6,942,518; 7,041,468; 7,167,818; and 7,299,082; U.S. Published Application Nos. 2004/0186365, now U.S. Pat. Nos. 7,811,231; 2005/0182306, now U.S. Pat. Nos. 8,771,183; 2006/0025662, now U.S. Pat. Nos. 7,740,581; 2006/0091006; 2007/0056858, now U.S. Pat. Nos. 8,298,389; 2007/0068807, now U.S. Pat. Nos. 7,846,311; 2007/0095661; 2007/0108048, now U.S. Pat. Nos. 7,918,975; 2007/0199818, now U.S. Pat. Nos. 7,811,430; 2007/0227911, now U.S. Pat. Nos. 7,887,682; 2007/0233013; 2008/0066305, now U.S. Pat. No. 7,895,740; 2008/0081977, now U.S. Pat. Nos. 7,618,369; 2008/0102441, now U.S. Pat. Nos. 7,822,557; 2008/0148873, now U.S. Pat. Nos. 7,802,467; 2008/0161666; 2008/0267823; and 2009/0054748, now U.S. Pat. No. 7,885,698; U.S. patents application Ser. No. 11/461,725, now U.S. Pat. Nos. 7,866,026; 12/131,012; 12/393,921, 12/242,823, now U.S. Pat. Nos. 8,219,173; 12/363,712, now U.S. Pat. Nos. 8,346,335; 12/495,709; 12/698,124; 12/698,129, now U.S. Pat. Nos. 9,402,544; 12/714,439; 12/794,721, now U.S. Pat. No. 8,595,607; and Ser. No. 12/842,013, now U.S. Pat. No. 9,795,326; and U.S. Provisional Application Nos. 61/238,646, 61/246,825, 61/247,516, 61/249,535, 61/317,243, 61/345,562, and 61/361,374.

### BACKGROUND

The detection and/or monitoring of glucose levels or other analytes, such as lactate, oxygen, AlC, or the like, in certain individuals is vitally important to their health. For example, the monitoring of glucose level is particularly important to individuals with diabetes and those with conditions indicative of onset of diabetes. Diabetics generally monitor glucose levels to determine if their glucose levels are being maintained within a clinically safe range, and may also use this information to determine if and/or when insulin is needed to reduce glucose levels in their bodies or when additional glucose is needed to raise the level of glucose in their bodies.

With the development of glucose monitoring devices and systems that provide real time glucose level information in a convenient and pain-less manner, there is an ongoing desire to integrate such monitoring devices and systems into daily life and activities to improve glycemic control. More specifically, there is a strong desire to identify the impact of daily activities such as exercise, medication administration, meal consumption and so forth on glucose level fluctuation and provide actionable, personalized health related information to tightly control glycemic variations. Furthermore, there is a strong desire to provide accuracy in medication dose determination that accurately assess the correct medication dose determination while reducing errors in such determination by taking into consideration parameters that impact medication therapy in the daily activities including exercise and meal consumption.

### SUMMARY

Embodiments of the present disclosure include multi-phase glucose response pattern determination and dynamic adjustment or modification to personalize the glycemic response to the particular activities and external parameters relevant to a specific patient or user. In certain embodiments, an analysis module is provided as a software application (“App”) that is executable by any processor controlled device, and in particular, a smart phone with communication capabilities to receive, analyze, transfer, transmit, display or output actionable information, for example, including therapy recommendation based on the determined glucose response pattern. In certain embodiments, the glucose response pattern, determined in view of a particular activity or combinations of activities, meal intake, medication intake, or any other external parameters specific to the daily activities of a user or a patient, is intelligently and dynamically adjusted on an on-going real time basis as additional activity specific or external parameter specific data is received and analyzed by the App.

Embodiments of the present disclosure include an overall network with sensor based devices in communication with the smart phone configured to execute the App, and optionally a data communication network with one or more back-end server terminals providing a network cloud configuration that is configured to either execute the functions of the App for analysis, for example, when in direct data communication with the sensor based devices, and provide the results of the analysis to the smart phone, or configured to operate in a more passive role, such as performing data backup functions or data repository functions for the smart phone and/or the sensor based devices. Also, optionally included in the overall network are one or more medication devices such as an insulin pump or an insulin injector pen that is configured to receive analysis data from the smart phone, from the one or more back-end server terminals, or directly from the sensor based devices.

Embodiments of the present disclosure include a data collection phase during which user or patient specific information is collected from one or more of the sensor based devices, by manual user input, or from a medication delivery device, for example, over a predetermined time period. When it is determined that sufficient amount of information about the patient or the user as it relates to glucose response and glycemic variation (for example, a minimum of 5 days, 6 days, one week, 10 days, 14 days, or any one or more combination of the number of days or portions of days), the App executed on the smart phone in certain embodiments may prompt the user or the patient that a specific glycemic response pattern has been determined or identified and is ready for user input for response analysis. To reach this point, in certain embodiments, the App analyzes data or information from the sensor based devices and other received user or patient specific parameters, and categorizes the received data, as part of the data analysis to determine the glucose response pattern, and thereafter continuously and dynamically updates the response pattern with the additional real time information received from the one or more sensor based devices or other user or patient specific parameters. In this manner, in certain embodiments, when the user inputs an activity or a parameter that the user wishes to engage in (for example, a 90 minute run that includes approximately 1,000 feet of incline, or number of steps taken during an established time period such as 12 hours, 18 hours, 24 hours, or other suitable time periods), the App, using the dynamic glucose response pattern recognition capabilities, is configured to notify the user or the patient that such activity will result in a specific glucose response (for example, a reduction in the glucose level, post activity, of approximately 25 mg/dL).

Further, in certain embodiments, the App may be configured to provide recommendations in addition to the physical activity driven analysis performed, such as, for example, provide a list of food type and amount to be consumed at a particular time prior to engaging in the activity, and/or within a fixed time post-activity so as to minimize glycemic fluctuation exceeding a predetermined range over a set time period spanning from prior to the activity, during, and post activity. In certain embodiments, the App is configured to perform similar analysis described above with recommendations where instead of the physical activity to be performed, the analysis relates to the amount of medication, food, drink, or one or more combinations thereof, to be consumed. In this manner, in certain embodiments, the user or the patient can take actions before consuming food and/or drinks or administering medication.

These and other features, objects and advantages of the present disclosure will become apparent to those persons skilled in the art upon reading the details of the present disclosure as more fully described below.

### BRIEF DESCRIPTION OF THE DRAWINGS

FIG. 1 is an overall glucose response data analysis system in accordance with one embodiment of the present disclosure;

FIG. 2A is a block diagram of the analysis module of FIG. 1 in accordance with one embodiment of the present disclosure;

FIG. 2B illustrates the information flow in conjunction with the analysis module of FIG. 1 performing data categorization, pattern recognition and dynamic update in accordance with one embodiment of the present disclosure;

FIG. 3 is an exemplary screenshot of the data input interface 111 (FIG. 2A) in accordance with one embodiment of the present disclosure;

FIG. 4 is a flowchart illustrating a routine to determine the impact of day time activity on overnight glucose level in accordance with one embodiment of the present disclosure;

FIG. 5 is a flowchart illustrating another routine to determine the impact of day time activity on overnight glucose level in accordance with one embodiment of the present disclosure;

FIG. 6 is a flowchart illustrating glucose response pattern identification and characterization for a particular activity based on absolute overnight glucose level in accordance with one embodiment of the present disclosure;

FIG. 7 is a flowchart illustrating glucose response pattern identification and characterization for a particular activity based on day-to-night glucose level change in accordance with one embodiment of the present disclosure;

FIG. 8 is a flowchart illustrating glucose response pattern identification and characterization for a particular activity based on day-to-night glucose level ratio in accordance with one embodiment of the present disclosure;

FIG. 9 illustrates a process flow for training and notification in accordance with one embodiment of the present disclosure; and

FIG. 10 illustrates a process flow for training and notification in accordance with another embodiment of the present disclosure.

FIG. 11 is a graph of an exemplary line fit analysis in accordance with the one embodiment of the present disclosure.

FIG. 12 is a graph of an exemplary line fit analysis in accordance with the one embodiment of the present disclosure.

FIG. 13 is a graph showing R values in accordance with the one embodiment of the present disclosure.

FIG. 14 is a graph of an exemplary line fit analysis in accordance with the one embodiment of the present disclosure.

FIG. 15 is a graph of an exemplary line fit analysis in accordance with the one embodiment of the present disclosure.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges as also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

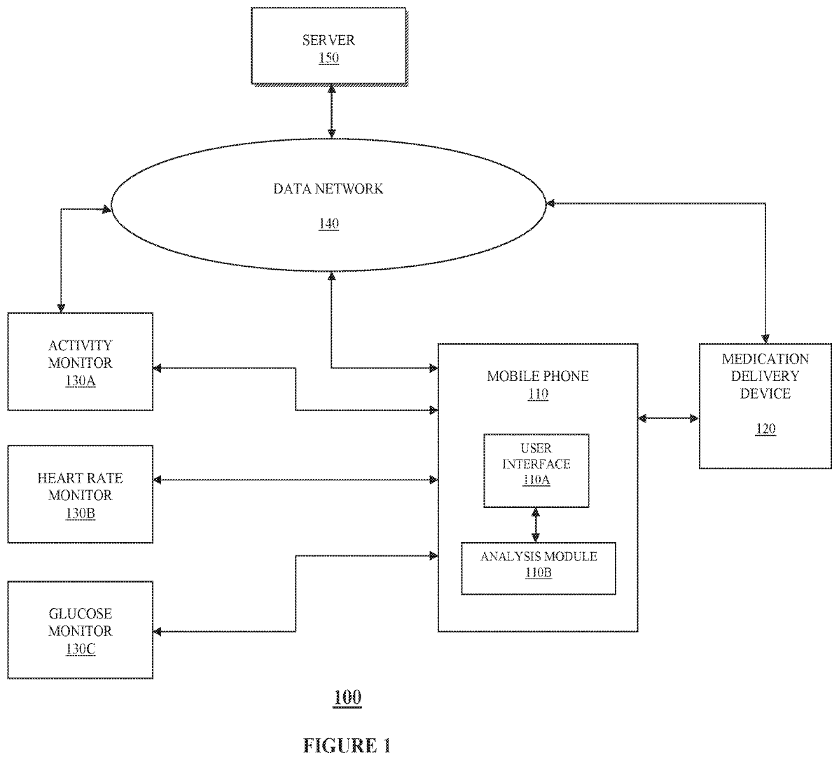

FIG. 1 is an overall glucose response data analysis system in accordance with one embodiment of the present disclosure. Referring to the Figure, glucose response data analysis system 100, in certain embodiments, includes a mobile phone 110 including user interface 110A and analysis module 110B programmed in the mobile phone 110 as an App, for example, installed as a downloaded executable file over data network 140 from server 150. As discussed in further detail below, in certain embodiments, data conditioning, analysis and dynamic glucose response pattern recognition and/or updating the glucose response pattern recognition is implemented as one or more executable routines by the App.

Referring back to FIG. 1, also shown are activity monitor 130A, heart rate monitor 130B, and glucose monitor 130C each in data communication with the mobile phone 110, or alternatively or in addition to, each in data communication with server 150 over data network 140. In this manner, each monitor 130A, 130B, 130C, in certain embodiments, is programmed to communicate the monitored information to server 150 for storage and/or analysis, or to mobile phone 110 for storage, analysis, and subsequent communication of either or both raw data received from each monitor 130A, 130B, 130C, and/or processed data or information from each monitor 130A, 130B, 130C to server 150 over data network for storage and/or further analysis.

Referring still to FIG. 1, also shown in glucose response data analysis system 100 is medication delivery device 120 in data communication with mobile phone 110, server 150, or one or more of the monitors 130A, 130B, 130C over data network 140. While not shown, in certain embodiments, the operation of the routines and functions of the App may be implemented in medication delivery device 120 where medication delivery device 120 directly receives data or information from one or more of the monitors 130A, 130B, 130C, and performs glucose response pattern recognition and analysis, and, for example, modifies a medication delivery profile (e.g., basal insulin delivery rate, determine a bolus insulin dose amount) based on the determined glucose response pattern from the monitored data (e.g., physiological monitored condition, and/or consumption of food and/or drinks, and medication intake) in view of the proposed physical activity and/or food or drink consumption.

In certain embodiments, mobile phone 110 includes one or more monitors 130A, 130B, 130C integrated within the phone 110. For example, mobile phone 110, in certain embodiments, includes an accelerometer and/or gyroscope that can monitor the movement of the mobile phone 110 user, such as keeping track or recording the number of steps taken, physical activities engaged (while having the mobile phone 110 on or close to the body such as using an arm band) such as number of steps taken, runs, jogs, sprints, each with a degree or level of intensity. In certain embodiments, mobile phone 110 is provided as a wrist watch configuration in which case mobile phone 110 includes a heart rate monitor in addition to the accelerometer or the gyroscope. In certain embodiments with the mobile phone 110 configured as a wrist watch, the mobile phone 110 incorporates a glucose sensor—in vivo, dermal, transdermal, or optical, such that the real time monitoring function of the glucose level is incorporated into the mobile phone 110.

Referring still again to glucose response data analysis system 100, in certain embodiments, a hub device (not shown) may be incorporated into the system 100, which is configured to communicate with one or more of the monitors 130A, 130B, 130C for data reception, storage, and subsequent communication to other devices in the system 100 over data network 140, or in direct communication with other devices in the system 100 such as, for example, mobile phone 110 and/or medication delivery device 120. The hub device, in certain embodiments, is configured as a pass through relay device or adapter that collects information from one or more of the monitors 130A, 130B, 130C, and either in real time or after a certain time period of data collection, transfers or sends the collected data to server 150, to mobile phone 110, and/or to medication delivery device 120. In certain embodiments, hub device is physically embodied as a small, discreet key fob type or dongle type device which the user or the patient keeps close to the body and communicates directly with monitors 130A, 130B, 130C worn on the body. Further, while three monitors 130A, 130B, 130C are shown in glucose response data analysis system 100, within the scope of the present disclosure additional sensors are provided to monitor other or related parameters of the user. For example, parameters for monitoring or measuring by one or more sensors include, but are not limited to, perspiration level, temperature level, heart rate variability (HRV), neural activity, eye movement, speech, and the like. Each one or more of these monitored parameters in certain embodiments of glucose response data analysis system 100 is used as input parameter to the analysis module 110B of mobile phone 110 as discussed in further detail below.

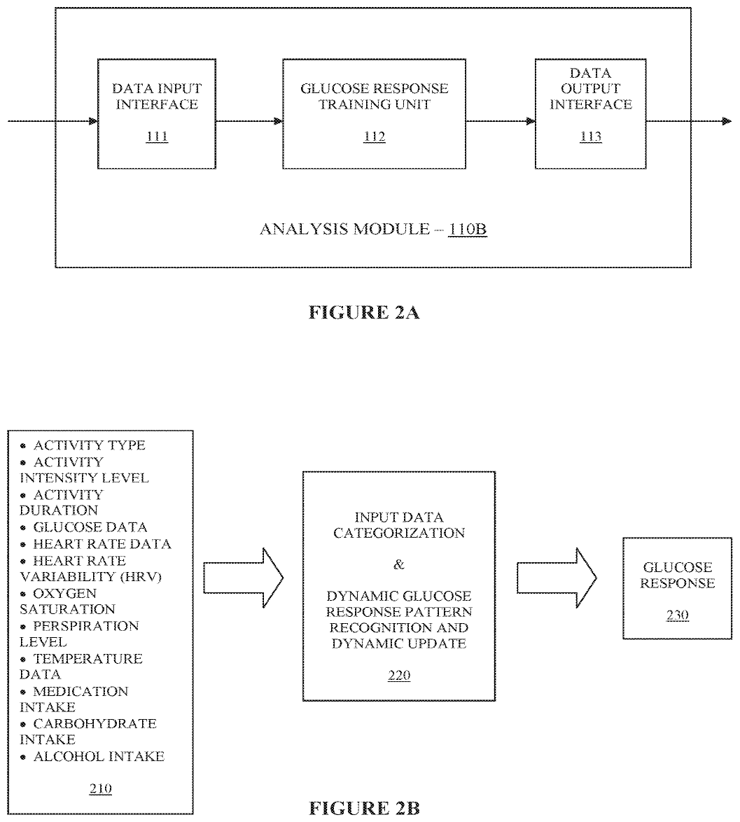

FIG. 2A is a block diagram of the analysis module 110B of FIG. 1 in accordance with one embodiment of the present disclosure. As shown in certain embodiments, analysis module 110B of mobile phone 110 includes data input interface 111 for interfacing or receiving data input from one or more 130A, 130B, 130C monitors external to mobile phone 110 or internal and within mobile phone 110. Data and/or information received via data input interface are provided to glucose response training unit 112. In certain embodiments, glucose response training unit 112 categorizes the received input data into respective categories depending upon the type of data, and the type or types of parameter associated with the data. For example, if the type of data is associated with a physical activity such as a 90 minute run, the parameters associated with the data include, in addition to duration, the level of run intensity (run, jog, sprint) which, in certain embodiments, may be determined using monitored heart rate information (if available) or pace of the run, aerobic or anaerobic run, competitive or non-competitive (training) run, or any other suitable category associated with the physical activity (e.g., the run). In certain embodiments, other type of data associated with the physical activity can be used such as number of steps taken during an established time period.

With the categorized data received from the one more monitors 130A, 130B, 130C (FIG. 1), the time corresponding glucose level information is retrieved (or received from glucose monitor 130C (FIG. 1)), and glucose response training unit 112 performs dynamic glucose response pattern recognition based, for example, on the analysis tools provided in the App for execution on mobile phone 110. Further, in certain embodiments, glucose response training unit 112 is configured to dynamically and continuously update the determined glucose response pattern based on the real time information from the one or more monitors (FIG. 1).

In certain embodiments, the accuracy of the glucose response pattern improves with increased data set over a longer time period (and/or with higher resolution/monitored frequency). However, a person's glycemic response to inputs may change over time. Certain embodiments address this by “resetting” or clearing the data set after some predetermined time period has elapsed. In other embodiments, the App recognizes that exceeding a set data collection duration potentially introduces error in accuracy of the glucose response pattern, in which case, when this point in time has reached, the App is configured to reset and enter the data collection period during which user driven analysis of glucose response feedback is disabled for at least the minimum number of days or hours for which monitored data is necessary to analyze and determine a new glucose response pattern. As described in further detail below, in certain embodiments, the App is configured to establish a “forgetting” window during which user driven analysis of glucose response feedback is continuously updated. The “forgetting” window, in certain embodiments, includes one or more of a predetermined time period set by the App or based on user input, or alternatively, is dynamically modified based on the glucose response feedback.

Referring back to FIG. 2A, in certain embodiments, the output of glucose response training unit 112 is provided to data output interface 113 which is operatively coupled to user interface 110A of mobile phone 110 for display, output or otherwise notification or prompt to the user of mobile phone 110 that the App has completed the initial or preliminary analysis and is operational to analyze glucose response to inputs such as number of steps taken, bike rides, runs, hikes, meals, for which the user or patient wishes to identify the corresponding glucose response so as to take timely action (corrective or proactive) to maintain glycemic control and minimize undesirable glucose fluctuations.

FIG. 2A illustrates the information flow in conjunction with the analysis module 110B of FIG. 1 performing data categorization, pattern recognition and dynamic update in accordance with one embodiment of the present disclosure. Referring to FIG. 2A, in certain embodiments, analysis module 110B of mobile phone 110 (FIGS. 1, 2A) executing the App is configured to categorize (220) the received input data (210), such as for example, type of activity, intensity level, duration, location, altitude information, glucose level, heart rate information, heart rate variability (HRV) information, oxygen saturation level, perspiration level, temperature level, medication intake information, type of medication, medication administration duration, time of day information corresponding to the administration of medication, carbohydrate intake information, alcohol consumption information or any other related metric for the particular monitored condition corresponding to the input data received.

With the received information, in certain embodiments, glucose response training unit 112 (FIG. 2A) performs dynamic glucose response pattern recognition and updates to the pattern (220) as new or additional data is received. As discussed in further detail below, in certain embodiments, prior to the output of the glucose response profile (230) based on the determined pattern, glucose response training unit 112 of analysis module 110B in mobile phone 110 ensures that sufficient input data has been analyzed. Once this point has reached and monitored information over at least a minimum time duration has been received and analyzed, the App, in certain embodiments, is configured to generate a notification to the user (for example, as an output prompt on the user interface 110A of mobile phone 110) when it determines information that may be useful to the user. Notifications may be made automatically, such as an alarm notification; or retrieved by the user when using the App, such as accessing the information from a menu; or displayed when the user next interacts with the App. An example of useful information is that the user's glucose levels are typically 20% lower overnight after they exercise during the prior day. The user can use this information to make sure that they do not experience night time hypoglycemia, for instance, by reducing their insulin coverage during this time or by having a snack before bedtime.

In another aspect of the present disclosure, the App prompts the user to enter contextual information when it detects certain conditions that warrant more information to be entered. The information entered is used by the routine that analyzes the input data to determine glycemic response patterns. The App contains routines that detect conditions, for instance, when meals have occurred or when activity has occurred, and notifies the user when these conditions are detected. Embodiments of the user notification includes one or more of an icon display, auditory or text output notification, or vibratory notification configured to prompt the user to provide more information about the condition that was detected. Examples of the one or more conditions include detected movement, detected rate of change of glucose increase or decrease exceeding or accelerating beyond a set threshold, detected spike or change in heart rate, perspiration or temperature level. Alternatively, rather than an alarm type notification, the App may provide the notification when the user next interacts with the App or the smartphone.

Referring yet again to the Figures, glucose response training unit 112 of analysis module 110B, in certain embodiments, is configured to perform dynamic glucose response pattern recognition based on glucose metrics that characterize the impact of a particular activity or event for a specific user or a patient, for example, impact of a particular activity or event (meal or medication intake, for example) for specific time of day periods that occur during and after an activity. Different glucose metrics such as mean or median glucose level can be used as the glucose metric. In certain embodiments, the use of median glucose information is less susceptible to outlier glucose data as compared to mean glucose level.

In certain embodiments, the glucose response training unit 112 determines the median of the continuously monitored glucose level during an overnight period after a particular activity, such as from 10 μm to 3 am, or from 3 am to 8 am, or from 10 μm to 8 am, for example. In certain embodiments, the glucose response training unit 112 uses the median glucose level determined during the day time periods, such as from 8 am to 10 pm, from 8 am to 6 pm, from 9 am to 5 pm, from 5 μm to 10 μm, or any other suitable day time period ranges. In certain embodiments, the median glucose information is determined with reference to a particular activity such that the median glucose level is determined for period of time after the start of the activity (2 hours after start of activity) for specific time duration (e.g., 12 hours). In certain embodiments, the relative start time for determining median glucose level and the duration of time period varies depending on the type of activity and/or other parameters related to the activity or associated with the user or the patient.

While the embodiments disclosed focus on activity during the daytime period impacting glucose levels at night, within the scope of the present disclosure similar analysis applies to any time periods defined by fixed times-of-day, such as activity in the morning (e.g., 5 am to 12 pm) impacting glucose levels post-dinner (e.g., 6 pm to 10 pm). Alternatively, the analysis disclosed herein within the scope of the present disclosure is applied to periods defined by events that occur regularly. For instance, the activity data set are generated from time periods defined each day as 5 am to breakfast where breakfast is a different time every day and determined by a user-entered or generated indication, or by an algorithm that processes glucose data to determine meal starts or by a recorded rapid acting insulin infusion. Exemplary embodiments of algorithmically detecting meal starts are disclosed in WO 2015/153482 (having International Application No. PCT/US2015/023380, filed Mar. 30, 2015), assigned to the Assignee of the present application, and the disclosure of which is incorporated by reference in its entirety for all purposes.

Further, the impacted time period may be defined likewise as the time period starting at when a meal is detected, such as the start of dinner until midnight. Also, within the scope of the present disclosure, a hybrid approach is provided where the activity time period is determined as a fixed time-of-day period while the impacted time period is determined by particular meal start times. Within the scope of the present disclosure, the impact on multiple time periods, such as post-breakfast, post-lunch, post-dinner and overnight are included. Further, the analysis can be extended to time periods across multiple days; for instance, determining how an activity occurring in a morning period of a first day impacts glucose levels on a subsequent day.

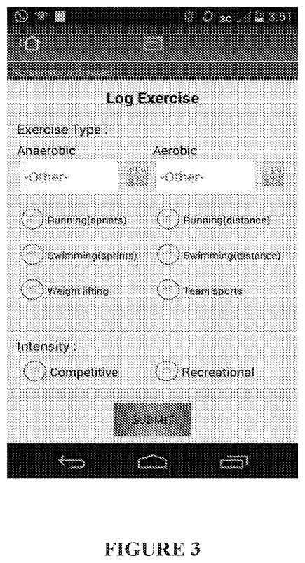

In addition, within the scope of the present disclosure two or more activity types can be used for analysis. A nonlimiting example requires a) users to enter into the user interface (UI) of the App (e.g., data input interface 111 of analysis module 110B (FIG. 2A)) contextual information related to the activities they perform, or b) using one or more sensors to differentiate between different types of activities, or c) alternative detection technology to differentiate between different types of activities. For user entered information approach (a) above, the App is configured to present a user interface (as shown in FIG. 3, for example) to allow users to enter activity information. In certain embodiments, users can enter information from a checklist or free-text entry. In addition, the App is configured to detect when measured activity exceeded a predefined threshold and prompt the user to enter this information. For the approach using one or more sensors to detect different activities (approach (b)), a combination of pedometer, heart rate sensor, and location sensor can be used where one or more thresholds and defined logic are configured to identify body motion, intensity, and speed and altitude change. Finally, for the approach using alternative detection technology (approach (c)), a location sensor may be used, for instance, to detect when the user is at the weightlifting gym, so that activity measured can be associated with anaerobic activity.

When an activity type attribute is associated with a measured activity metric, the analysis described below can be performed for each activity type. For example, if two activity types are used, such as aerobic and anaerobic, the analysis described below can be used to determine the impact of aerobic activity on future glucose levels, and independently determine the impact of anaerobic activity on future glucose levels. Within the scope of the present disclosure, one or more combinations of activities and analysis time periods can be achieved such as days with both types of activity indicating a new type of activity.

In certain embodiments, glucose response training unit 112 determines glucose median level, activity and other related parameters for multiple daytime periods and median glucose level is determined for associated overnight periods that follow the daytime periods. In certain embodiments, glucose response training unit 112 determines glucose median levels for the time of day periods for days without activity. More specifically, glucose response training unit 112, in certain embodiments, is configured to confirm with the user or patient that significant activity (e.g., an exercise event, number of steps taken during a day time period (12 hours, 18 hours, 24 hours, or other suitable time periods), a run, bike ride, hike, etc.) did not occur during these days without significant activity. With time periods separated between those days with significant activity and those days without significant activity, glucose response training unit 112, in certain embodiments, analyzes the received input data (see FIG. 2A) to characterize the impact of particular activities on overnight glucose level to generate the dynamic glucose response pattern—that is, to assess how the user or patient's body reacts to the specific activities, and to generate or provide appropriate therapy recommendation to the user or the patient when the user decides to engage in the same activities with the same or similar parameters such as duration, level of intensity and the like.

FIG. 3 is an exemplary screenshot of the data input interface 111 (FIG. 2A) in accordance with one embodiment of the present disclosure. Referring to FIG. 3, in certain embodiments, customized data entry screen is presented to the user for information entry for analysis by the App. In a nonlimiting example, a set of radio buttons on the user interface (of the mobile telephone executing the App, for example) are seeded with one or more default activity related parameters such as number of steps, run, jog, hike, bike ride, swim, sleep, and/or food/drink related parameters such as coffee, alcohol with sugar, alcohol without sugar, cereal, bacon, toast, and the like, with the option to modify over time as new custom answers/feedback or responses are added by the user. This allows the user to quickly enter the most common or most used types of activity without losing the flexibility to enter other types of custom data.

Within the scope of the present disclosure, the App provides multiple means for users or patients to enter information about meals and activity. The patient can proactively enter this information. This is particularly useful for meal entry where a photo of the meal can be entered. This may be a much more convenient and fun way for users or patients to enter and view meals information. Additional details can be found in Provisional Patent Application No. 62/307,344 entitled “Systems, Devices, and Methods For Meal information Collection, Meal Assessment, and Analyte Data Correlation” filed concurrently herewith. As discussed above, in certain embodiments, the App may detect a meal or activity episode and prompt the patient for more information as disclosed in WO 2015/153482 incorporated by reference in its entirely for all purposes.

For users or patients that use insulin or take other glucose-altering medications, the App may be configured to automatically retrieve user/patient specific data regarding use of these medications or allow manual patient entry into the system.

Within the scope of the present disclosure, the App is configured to facilitate experimentation and understanding by providing a meal/activity analysis output. In certain embodiments, the output is presented as one or more reports on the smartphone or on a web browser retrieved from a server. The one or more reports list meal episodes as defined by glucose excursions. The list of meal episodes can be sorted by date-time of the episode, or by severity of the glucose excursion, such as measured by peak glucose level, by glucose change over the course of the excursion, or by area defined by glucose and duration of the excursion. Each row in the analysis output report(s) includes information associated with the meal episode. In certain embodiments, the report(s) includes one or more of the photos or otherwise text entries associated with that meal episode, date-time, and one or more meal severity metrics. The report(s), in certain embodiments, also includes any related activity information within some period of time of the meal. Too much information on this list may be too cluttered to be practical. Thus, the App, in certain embodiments, provides the user or the patient to manipulate the presentation of information, such as selecting the row and presenting a popup window with a more detailed information screen. Such detailed information screen also provides a glucose plot associated with the meal episode. In this manner, meals that have the most impact on glucose levels can be highlighted in an easy to view presentation to provide a better understanding of the impact of certain foods on their glucose levels so that the user or the patient can avoid or limit foods that are detrimental to their health.

The App, in certain embodiments, is also configured to learn how food and activity can impact future glucose levels. When food and activity are selected on the customizable checklist described above, glucose data are associated with these selections and multiple glucose datasets can be associated with a single entry type. Also, multiple glucose datasets can be associated with combinations of one or more meal entry types and one or more activity entry types. The glucose datasets may be processed in one or more different manners in order to characterize the impact of the episode on glucose levels.

In certain embodiments, the median glucose levels from all of the data sets are determined and compared to the median of all periods of captured glucose data. Alternatively, this approach can be applied to individual time-of-day periods, such as pre-breakfast, post-breakfast, post-lunch, post-dinner and post-bedtime. Over time, the App is configured to estimate with some level of confidence the glycemic impact for any given entry type or combination of entry types. For instance, a specific activity type “bike ride uphill” for 1 or more hours of activity may be associated with a 20% increase in patient insulin sensitivity for the next 24 hours—the change in insulin resistance is readily associated with the change in median glucose. This association may be made by the system when the system detects that the statistical level of confidence has exceed some predetermined amount. This information may alter the parameters used in bolus calculator over the next 24 hours. Alternatively, the App may detect activity associated with the bike ride and alert the patient, for instance, at bedtime so they can have a snack to avoid hypoglycemia that night.

Another type of output report presented by the App includes a list of activities that can be sorted by median glucose levels over the period of time following the activity, such as 24 hours. The list can illustrate which activities have the biggest impact on future glucose levels. Further, another type of report can present a list of food and activity combinations, in the same way as described. These approaches can be readily extended to other sensor data and other contextual inputs, such as illness, alcohol consumption, coffee consumption, and the like.

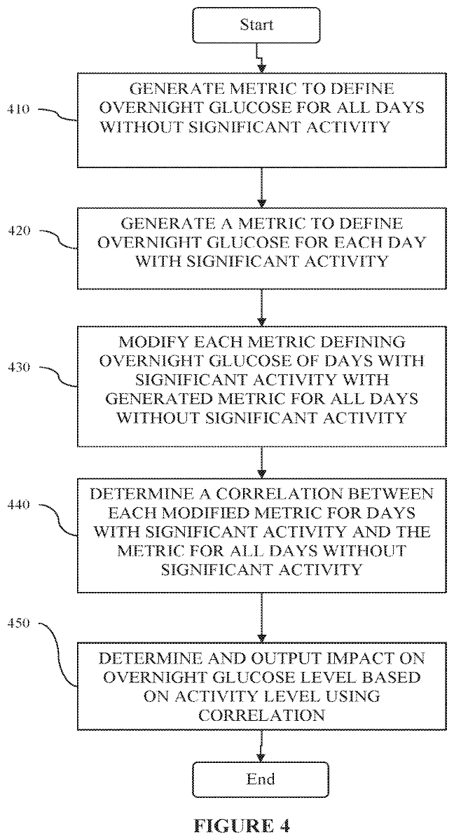

FIG. 4 is a flowchart illustrating a routine to determine the impact of day time activity on overnight glucose level in accordance with one embodiment of the present disclosure. Referring to FIG. 4, in one embodiment, determining the impact of day time activity on overnight glucose level includes generating a metric to define an overnight glucose level for all days without significant activity over a predetermined time period (e.g., 2 weeks, a month, or any other suitable time period) (410). Thereafter, a metric is generated to define the overnight glucose level for each day with significant activity in the predetermined time period (420). Within the scope of the present disclosure the determination of days with or days without significant activity is based on one or more activity metric exceeding a defined threshold (e.g., number of steps exceeding a threshold within a 24 hour time period). Referring back to FIG. 4, after generating the metric to define overnight glucose level for all days without significant activity, and a plurality of metrics to define the overnight glucose level for each day with significant activity, each of the plurality of metrics to define the overnight glucose level for each day with significant activity is modified with the metric for all days without significant activity (430). Then, a correlation is determined between each modified metric for days with significant activity and the metric for all days without significant activity (440), and thereafter, given an activity level, the impact on the overnight glucose level of the activity level is determined and presented to the user based on the determined correlation (450).

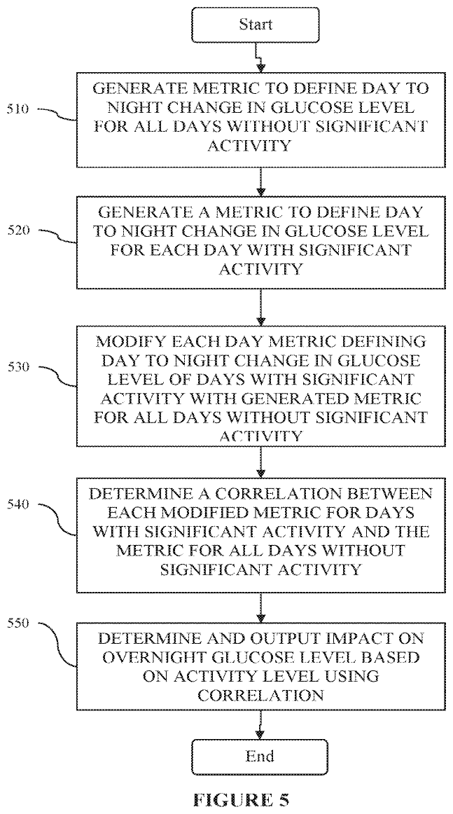

FIG. 5 is a flowchart illustrating another routine to determine the impact of day time activity on overnight glucose level in accordance with one embodiment of the present disclosure. Referring to FIG. 5, in one embodiment, determining the impact of day time activity on overnight glucose level includes generating a metric to define a day-to-night change in glucose level for all days without significant activity over a predetermined time period (for example, 2 weeks, a month, or other suitable time periods) (510). Thereafter, a plurality of metrics is generated to define day-to-night change in glucose level for each corresponding day with significant activity (520). With a metric for day-to-night change in glucose level for each day with significant activity and a metric for day-to-night change in glucose level for all days without significant activity, each day metric defining day-to-night change in glucose level for days with significant activity are modified with the metric for day-to-night change in glucose level for days without significant activity (530). Then, a correlation relationship is determined between each modified metric for days with significant activity and the metric for all days without significant activity (540). With the determined correlation, for a given activity level, the impact of the activity level on the overnight glucose level based on the determined correlation is determined and presented to the user (550).

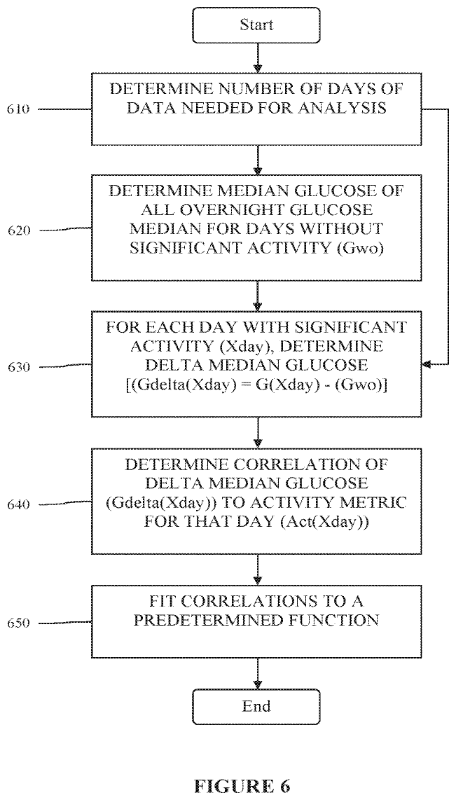

FIG. 6 is a flowchart illustrating glucose response pattern identification and characterization for a particular activity based on absolute overnight glucose level in accordance with one embodiment of the present disclosure. Referring to FIG. 6, based on the input data received from one or more of the monitors 130A, 130B, 130C, glucose response training unit 112 of analysis module 110B (FIG. 2A) determines whether sufficient amount of data has been received via data input interface 111 (FIG. 2A). In certain embodiments, the amount of data sufficient to perform the glucose response pattern and characterization analysis is based on data received over a predetermined number of days with significant activity, and a predetermined number of days without significant activity (collectively, “X”). In certain embodiments, whether a particular activity qualifies as significant activity is determined based on one or more of activity duration, calories burned during the duration of the activity, the level of intensity of the activity, whether the activity is aerobic or anaerobic activity, or type of activity (for example, competitive activity or non-competitive, training activity). For example, glucose response training unit 112, in certain embodiments, determines that input data from one or more monitors 130A, 130B, 130C (FIG. 1) for 3 days with significant activity and 3 days without significant activity provides the sufficient amount of data for analysis.

In an alternative embodiment, the determination of data sufficiency is based on the degree of certainly of the estimated glycemic pattern, rather than a predetermined number of days of data or amount of data.

Referring to FIG. 6, with the number of days of input data needed for analysis determined (610), glucose response training unit 112 (FIG. 2A) determines median glucose level of all overnight glucose median levels for the determined number of days without significant activity (Gwo) (620). In certain embodiments, number of days without significant activity (Gwo) is defined as the number of days during which the activity measure is below a predefined threshold, such as 10,000 steps during the predetermined day-time period (12 hours, 18 hours, or other suitable time periods). In certain embodiments, the median glucose level of all overnight glucose median levels for the number of days without significant activity (Gwo) varies depending upon the type of activity.

Thereafter, as shown in FIG. 6, for each day with significant activity (Xday), a delta median glucose level (Gdelta(Xday)) is determined (630), where delta median glucose level (Gdelta(Xday)) is the difference between the overnight glucose median for the particular day with significant activity G(Xday) and the median glucose level of all overnight glucose median levels for the determined number of days without significant activity (Gwo). That is:

$$( Gdelta ( Xday ) ) = G ( Xday ) - ( Gwo )$$

In certain embodiments, median glucose level of all overnight glucose median levels for the determined number of days without significant activity (Gwo) (620) and delta median glucose level (Gdelta(Xday)) for each day (630) are simultaneously determined. In other words, steps 620 and 630 can be performed serially, or in parallel relative to each other.

Referring still to FIG. 6, a correlation relationship between the median glucose level for the day (Xday) with significant activity (Gdelta(Xday)) and activity metric (Act (Xday)) for that day is determined (640), and the correlations are fit to a predetermined function (650). In certain embodiments, the correlation relationship includes a linear function, where the delta median glucose level for the days with significant activity (Gdelta(Xday)) is a linear function of the activity metric (Act(Xday)). Within the scope of the present disclosure, the correlation relationship includes a constant offset relationship, an exponential relationship, a logarithmic relationship, or a polynomial relationship, between the delta median glucose level for days with significant activity (Gdelta(Xday)) and the activity metric (Act(Xday)).

In certain embodiments, activity metric (Act (Xday)) is predetermined for the particular activity that the user or the patient engaged in and is based on, for example, input data categorization 220 (FIG. 2B) performed by glucose response training unit 112 of analysis module 110B. (FIG. 2A). In certain embodiments, activity metric (Act (Xday)) varies depending on one or more parameters associated with the activity including, for example, activity duration, intensity level, activity type, heart rate data associated with the activity, among others. In certain embodiments, the activity metric (Act(Xday)) includes a “step-rate” such as steps-per-hour, or steps over a predetermined or fixed time duration.

In certain embodiments, least squares technique is applied to fit the correlation relationship to the data set. For example, least squares approach can be applied to the data set to determine the slope and offset for the linear relationship defining the correlation between the delta median glucose level for days with significant activity (Gdelta(Xday)) and the activity metric (Act(Xday)). In certain embodiments, the linear relationship is subsequently applied by the App to predict or anticipate the impact of significant exercise on over-night glucose levels. In other words, with a known or determined activity metric (Act(Xday)), the App estimates the resulting delta median glucose level for days with significant activity (Gdelta(Xday)) by multiplying the activity metric (Act(Xday)) by the slope of the linear correlation relationship and adding the offset, where the slope and offset are parameters determined by a best fit analysis, for example. In certain embodiments, the best fit analysis is updated with each revision or addition of the data set collected or received from monitors (130A-130C FIG. 1). Alternatively, in certain embodiments, the best fit analysis is updated after a predetermined time period of data set collection.

In certain embodiments, a set of ratios (R) determined for each day with significant activity is determined. The ratios are calculated as the delta median glucose level for days with significant activity (Gdelta(Xday)) divided by the activity metric (Act(Xday)). The median or mean of the set of ratios are then calculated. The impact of the activity is then determined by multiplying the median of the set of ratios (R) times the current activity metric (Act(Xday)). Alternatively, within the scope of the present disclosure, curve fitting approach is applied such as using least squares technique to fit the set of ratios (R's) to a least squares fit line, for example.

Referring back to FIG. 6, in certain embodiments, the number of days needed for analysis (610) can be determined by the quality of the correlation (650). For example, in certain embodiments, linear line fit analysis provides metrics that indicate the quality of such line fit (for example, correlation coefficient (R²) or standard error of the delta median glucose level for days with significant activity (Gdelta(Xday)) estimate). The data set, in certain embodiments, is determined to be sufficient (610) if the line fit quality metric exceeds a specific value, for example (but not limited to) when the R² value is greater than 0.9, or the standard error of the delta median glucose level for days with significant activity (Gdelta(Xday)) for the line fit is less than 10%. If the line fit is determined to be invalid, in certain embodiments, the App is configured to continue with analysis of the data set (i.e., continue training), and each day the line fit is updated to determine if it is valid. When the line fit is determined to be valid, then the analysis result, in certain embodiments, is presented to the user, for example, at the data output interface 113 of analysis module 110B (FIG. 2A).

By way of a nonlimiting example, Table 1 below illustrates data set collected for glucose response pattern identification and characterization using number of steps taken as activity in accordance with certain embodiments of the present disclosure.

TABLE 1
14 days of activity vs nonactivity data

| | | Activity Metric | Daytime Median | Overnight Median |
|---|---|---|---|---|
| Day | Activity? | (steps) | Glucose (mg/dL) | Glucose (mg/dL) |
| 1 | yes | 12503 | 143 | 117 |
| 2 | no | 3043 | 156 | 142 |
| 3 | no | 2043 | 142 | 150 |
| 4 | yes | 11432 | 150 | 125 |
| 5 | yes | 16490 | 146 | 111 |
| 6 | yes | 13083 | 151 | 120 |
| 7 | no | 1044 | 143 | 160 |
| 8 | no | 1453 | 145 | 151 |
| 9 | yes | 10984 | 149 | 131 |
| 10 | no | 2354 | 139 | 140 |
| 11 | no | 2356 | 161 | 139 |
| 12 | no | 1234 | 155 | 144 |
| 13 | yes | 19245 | 144 | 105 |
| 14 | no | 7034 | 147 | 143 |

From Table 1 above, it can be seen that over the two week period, there were 6 days with activity (determined as number of steps exceeding a threshold level—e.g., 10000 steps taken within a 24 hour period) including days 1, 4, 5, 6, 9, and 13. It can also be seen that during the two week period, there were 8 days without activity (determined as the number of steps below the threshold level of 10000 steps within a 24 hour period) including days 2, 3, 7, 8, 10, 11, and 12.

Given the daytime median glucose level for each of the 14 days and also the corresponding overnight median glucose level for each of the 14 days, the median glucose level of all overnight median glucose level for days without significant activity (Gwo) is determined by taking the median of the overnight median glucose level of days 2, 3, 7, 8, 10, 11, and 12 from Table 1, which is 143.5 mg/dL. Further, for each day with activity (e.g., days 1, 4, 5, 6, 9, and 13), the delta median glucose (Gdelta(Xday)) is determined by subtracting median glucose level of all overnight median glucose level for days without significant activity (Gwo) determined as 143.5 mg/dL from the corresponding overnight median glucose level (G(Xday)). For example, for day 1 (activity), the delta median glucose (Gdelta(day1)) is 117 mg/dL subtracted by 143.5 mg/dL (median glucose level of all overnight median glucose level for days without significant activity (Gwo)) results is the delta median glucose (Gdelta(day1)) of −26.5. Similarly, for day 4 (activity), the delta median glucose (Gdelta(day4)) is −18.5 (125 mg/dL subtracted by 143.5 mg/dL). For day 5 (activity), the delta median glucose (Gdelta(day5)) is −32.5 (111 mg/dL subtracted by 143.5 mg/dL). For day 6 (activity), the delta median glucose (Gdelta(day6)) is −23.5 (120 mg/dL subtracted by 143.5 mg/dL). For day 9 (activity), the delta median glucose (Gdelta(day9)) is −12.5 (131 mg/dL subtracted by 143.5 mg/dL). Finally, for day 13 (activity), the delta median glucose (Gdelta(day13)) is −38.5 (105 mg/dL subtracted by 143.5 mg/dL).

With the delta median glucose for each day with activity (Gdelta(Xday)) determined as described above, a corresponding R value for each day with activity is determined by dividing the determined delta median glucose (Gdelta(Xday)) with the activity metric (Act(Xday)) for the corresponding day with activity. For example, R value for day 1 is −0.002 (−26.5 divided by 12,503 steps (activity metric for day 1). In this manner, the R value for the days with activity is determined and the resulting values are shown as below in Table 2 (with the corresponding delta median glucose level (Gdelta(Xday)).

TABLE 2

| | | | Overnight | Delta | |
|---|---|---|---|---|---|
| | | Activity | Median | Median | |
| | | Metric | Glucose | Glucose | |
| Day | Activity? | (steps) | (mg/dL) | (Gdelta) | R |
| 1 | yes | 12503 | 117 | −26.5 | −0.002119491 |
| 4 | yes | 11432 | 125 | −18.5 | −0.001618265 |
| 5 | yes | 16490 | 111 | −32.5 | −0.001970891 |
| 6 | yes | 13083 | 120 | −23.5 | −0.001796224 |
| 9 | yes | 10984 | 129 | −14.5 | −0.001320102 |
| 13 | yes | 19245 | 105 | −38.5 | −0.00200052 |

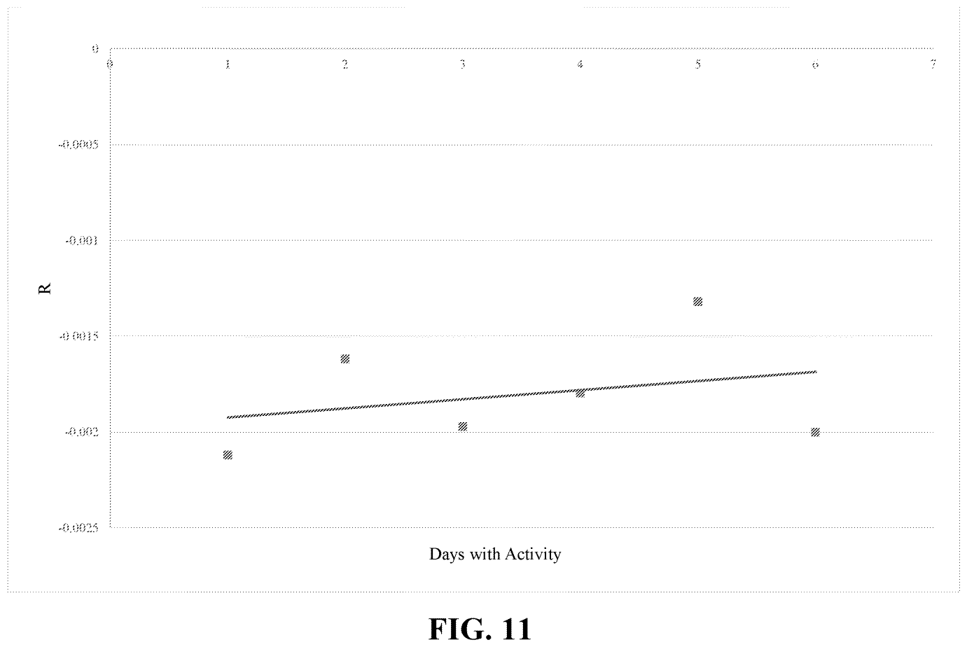

Based on the data set determined as shown in Table 2 above, a line fit analysis is performed on the days with activity against the corresponding R values as shown in FIG. 11.

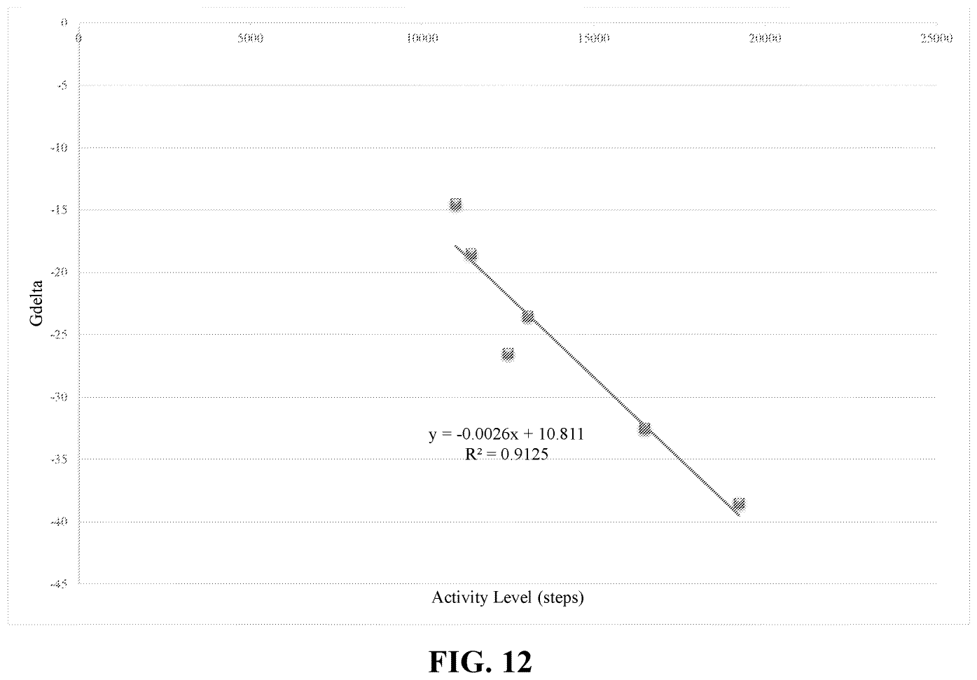

Alternatively, the median or mean of the R values can be used to represent the glycemic pattern. Further, a line fit analysis can be performed on the delta median glucose (Gdelta(Xday)) with respect to the activity level (number of steps) and as shown in FIG. 12 where it can be seen that the correlation value (R²) is 0.9125 demonstrating acceptable correlation, and where the line fit analysis provides an offset of 10.811 with a slope of −0.0026. This line represents the glycemic pattern.

Using FIG. 12, when the user decides to perform a particular activity that will result in 15,000 steps, from the line fit analysis, it can be seen that such activity will result in a reduction of the glucose level by approximately 28 mg/dL. With this information, if the user desires to maintain a tighter glycemic control, and knowing that performing 15,000 steps will reduce the glucose level by approximately 28 mg/dL, the user can take proactive actions to counter the effects of the activity (e.g., 15,000 step) by, for example, consuming more food and/or drinks either before or during engaging in the activity.

In an alternate embodiment, the activity metric is transformed into two values: significant activity or not significant activity. In this case, an overnight glucose median level is associated with either a day of significant activity or with a day without, where significant activity is defined as when the activity measure exceeds a predefined threshold (for example, the number of steps exceeding 10,000 steps for the day). More specifically, referring to Table 1, the median glucose for all overnight periods associated with days of significant activity are determined (days 1, 4, 5, 6, 9, and 13) as 118.5 mg/dL, as well as the median glucose level for all overnight periods associated with non-significant activity (days 2, 3, 7, 8, 10, 11, 12, and 14) as 143.5 mg/dL. Then, the decrease in median activity is determined by subtracting 143.5 mg/dL (as the median glucose level for all overnight periods associated with nonsignificant activity) from 118.5 mg/dL (the median glucose for all overnight periods associated with days of significant activity), which results in-25 mg/dL. The percentage median decrease is then 17.42% (−25 mg/dL divided by 143.5 mg/dL). In this approach, whether sufficient number of days of data set has been collected can be determined by using standard statistical tests for determining if the means of two different populations are different. For example, by confirming that the standard deviation of each median overnight glucose determination (with and without activity) is below a predefined threshold, such as 20 mg/dL, for example. Referring to Table 1, the standard deviation for days with significant activity (days 1, 4, 5, 6, 9, and 13) is 8.864 mg/dL, while the standard deviation for days without significant activity (days 2, 3, 7, 8, 10, 11, 12, and 14) is 7.08 mg/dL.

Referring again to the Figures, with the glucose response pattern identification and characterization described above, the App, in certain embodiment, is configured to output to the user when subsequent significant activity is detected: “For days with significant activity, overnight glucose levels tend to be 25 mg/dL lower, than for days without significant activity.” Alternatively, this result may be displayed as a percentage, for this example, 17% lower. Within the scope of the present disclosure, the technique described above can be expanded to any level of quantization such as three or four levels.

In certain embodiments, using the routine described above in conjunction with FIG. 6, glucose response training unit 112 of analysis module 110B (FIG. 2A) identifies consistent glucose response to a particular activity with specific parameters. The user or the patient then uses this information to modify or adjust therapy protocol, meals consumed or the type of activity to engage in given the underlying physiological state, to maintain tight glycemic control and improve health condition.

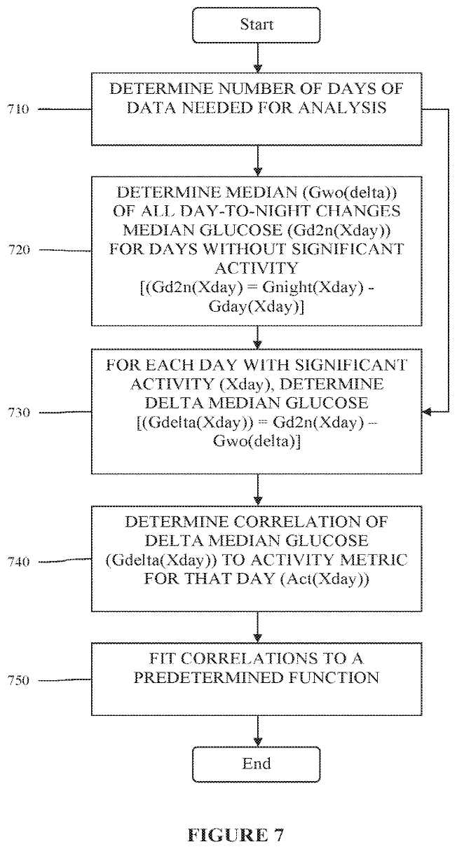

FIG. 7 is a flowchart illustrating glucose response pattern identification and characterization for a particular activity based on day-to-night glucose level change in accordance with one embodiment of the present disclosure. Referring to FIG. 7, similar to step 510 of FIG. 5, based on the input data received from one or more of the monitors 130A, 130B, 130C, glucose response training unit 112 of analysis module 110B (FIG. 2A), determines whether sufficient amount of data has been received via data input interface 111 (FIG. 2A) (710). Then, glucose response training unit 112 of analysis module 110B determines median (Gwo(delta)) of all day-to-night changes in glucose median (Gd2n(Xday)) for days (in the number of days determined to provide sufficient amount of data) without significant activity (720).

More specifically, each day-to-night changes in glucose median without significant activity (Gd2n(Xday)) is determined by subtracting the median glucose level over a first predetermined time-of-day period (e.g., from 8 am to 10 pm) (Gday(Xday)) from the median glucose level over a second predetermined time-of-day period (e.g., from 10 am to 6 pm) (Gnight(Xday)) (720). That is:

$$( G d 2 n ( Xday ) ) = Gnight ( Xday ) - Gday ( Xday )$$

Within the scope of the present disclosure the time periods and ranges for the first and second predetermined time-of-day periods may be varied so that one is longer than the other, or alternatively, the two periods are the same length. In certain embodiments, the first and second predetermined time periods for each day are determined based on specific events such as meal events or other indicators associated with the patient.

Referring back to FIG. 7, with the median of all day-to-night changes in median glucose for days without significant activity (Gwo(delta)) determined (720), glucose response training unit 112, in certain embodiments, determines delta median glucose level (Gdelta(Xday)) by subtracting median of all day-to-night changes in glucose median for days without significant activity (Gwo(delta)) from the day-to-night changes in glucose median without significant activity (Gd2n(Xday)) (730). In certain embodiments, determination of median of all day-to-night changes in median glucose for days without significant activity (Gwo(delta)) (720) and the delta median glucose level (Gdelta(Xday)) for each day with significant activity (730) are determined simultaneously rather than in sequence. In alternate embodiments, the delta median glucose level (Gdelta(Xday)) for each day with significant activity (730) may be determined before median of all day-to-night changes in median glucose for days without significant activity (Gwo(delta)) (720).

Thereafter, a correlation relationship is determined between delta median glucose (Gdelta(Xday)) and activity metric (Act (Xday)) for each day with significant activity (Xday) (740). Similar to the routine performed in conjunction with FIG. 6, in certain embodiments, activity metric (Act (Xday)) is predetermined for the particular activity that the user or the patient engaged in, and as such may be based on input data categorization (FIG. 2B) performed by glucose response training unit 112 of analysis module 110B. (FIG. 2A). Similarly, in certain embodiments, activity metric (Act (Xday)) varies depending on one or more parameters associated with the activity including, for example, activity duration, intensity level, activity type, heart rate data associated with the activity.

Again, similar to the routine executed in conjunction with FIG. 6, referring to FIG. 7, once the correlation relationship between the delta median glucose level for the day (Xday) with significant activity (Gdelta(Xday)) and activity metric (Act (Xday)) for that day is determined (740), the correlation relationship, for instance, where the delta median glucose level for days with significant activity (Gdelta(Xday)) is represented as a linear function of the activity metric (Act(Xday)), is used to generate an estimate of the delta median glucose level for days with significant activity (Gdelta(Xday)) of the next overnight period for days of significant activity, and the analysis result are displayed to the user. That is, the correlations are fit to a predetermined function (750) and the resulting relationship is output to the user.

For example, referring to the data set shown in Table 1, the median of all day-to-night changes in glucose median for days without significant activity (Gwo(delta)) is −1.5. This is derived from determining the median of all day-to-night changes in glucose median without significant activity (Gd2n(Xday)). That is, from Table 1, for each day without significant activity (days 2, 3, 7, 8, 10, 11, 12, and 14), the median day-to-night changes in glucose median (Gd2n(Xday)) is determined by subtracting the daytime median glucose level from the overnight glucose level. For example, the median of day-to-night changes in glucose median for day 2 (Gd2n(day2)) is −14 mg/dL (142 mg/dL-156 mg/dL). The median of day-to-night changes in glucose median for day 3 (Gd2n(day3)) is 8 mg/dL (150 mg/dL-142 mg/dL). The median of day-to-night changes in glucose median for day 7 (Gd2n(day7)) is 17 mg/dL (160 mg/dL-143 mg/dL). The median of day-to-night changes in glucose median for day 8 (Gd2n(day8)) is 6 mg/dL (151 mg/dL-145 mg/dL). The median of day-to-night changes in glucose median for day 10 (Gd2n(day10)) is 1 mg/dL (140 mg/dL-139 mg/dL). The median of day-to-night changes in glucose median for day 11 (Gd2n(day11)) is-22 mg/dL (139 mg/dL-161 mg/dL). The median of day-to-night changes in glucose median for day 12 (Gd2n(day12)) is −11 mg/dl (144 mg/dL-155 mg/dL). Finally, the median day-to-night changes in glucose median for day 14 (Gd2n(day14)) is −4 mg/dL (143 mg/dL-147 mg/dL). This is illustrated in Table 3 below.

TABLE 3

| | | | | | | Median of all |
|---|---|---|---|---|---|---|
| | | | | | | day-tonight changes |
| | | | Daytime | Overnight | Median | in glucose median |
| | | Activity | Median | Median | day-to-night | for days without |
| | | Metric | Glucose | Glucose | glucose change | significant activity |
| Day | Activity? | (steps) | (mg/dL) | (mg/dL) | Gd2n | Gwo(delta) |
| 2 | no | 3043 | 156 | 142 | −14 | |
| 3 | no | 2043 | 142 | 150 | 8 | |
| 7 | no | 1044 | 143 | 160 | 17 | |
| 8 | no | 1453 | 145 | 151 | 6 | |
| 10 | no | 2354 | 139 | 140 | 1 | |
| 11 | no | 2356 | 161 | 139 | −22 | |
| 12 | no | 1234 | 155 | 144 | −11 | |
| 14 | no | 7034 | 147 | 143 | −4 | −1.5 |

With the median of all day-to-night changes in glucose median for days without significant activity (Gwo(delta)) determined as −1.5, for each day with significant activity, the delta median glucose (Gdelta(Xday)) can be determined by subtracting the median day-to-night changes in glucose median for each day by the median of all day-to-night changes in glucose median for days without significant activity (Gwo(delta)). This is shown in table 4 below.

TABLE 4

| | | | Daytime | Overnight | Median | | |
|---|---|---|---|---|---|---|---|
| | | Activity | Median | Median | day-to-night | | |
| | | Metric | Glucose | Glucose | glucose change | Delta Median | |
| Day | Activity? | (steps) | (mg/dL) | (mg/dL) | Gd2n | Glucose Gdelta | R |
| 1 | yes | 12503 | 143 | 117 | −26 | −24.5 | −0.00195953 |
| 4 | yes | 11432 | 150 | 125 | −25 | −23.5 | −0.002055633 |
| 5 | yes | 16490 | 146 | 111 | −35 | −33.5 | −0.002031534 |
| 6 | yes | 13083 | 151 | 120 | −31 | −29.5 | −0.002254835 |
| 9 | yes | 10984 | 149 | 131 | −18 | −16.5 | −0.001502185 |
| 13 | yes | 19245 | 144 | 105 | −39 | −37.5 | −0.001948558 |

As can be seen from Table 4, for each day with significant activity, a corresponding R value is determined by dividing the determined delta median glucose (Gdelta(Xday)) with the activity metric (Act(Xday)) for the corresponding day with activity.

In addition, in certain embodiments, rather than a linear function, a set of ratios (R) determined for each day with significant activity is generated. The ratios R are determined by dividing delta median glucose (Gdelta(Xday)) for each day with significant activity by the corresponding activity metric (Act(Xday)). The median or mean of the set of ratios R is then determined (in this case, the median of the R values for days with significant activity is −0.00199553198802936). The effect of activity can then be determined by multiplying the median R by the current activity metric (Act(Xday)). Alternatively, curve fitting techniques can be applied using, for example, least squares to fit the set of ratios (R's) to a line.

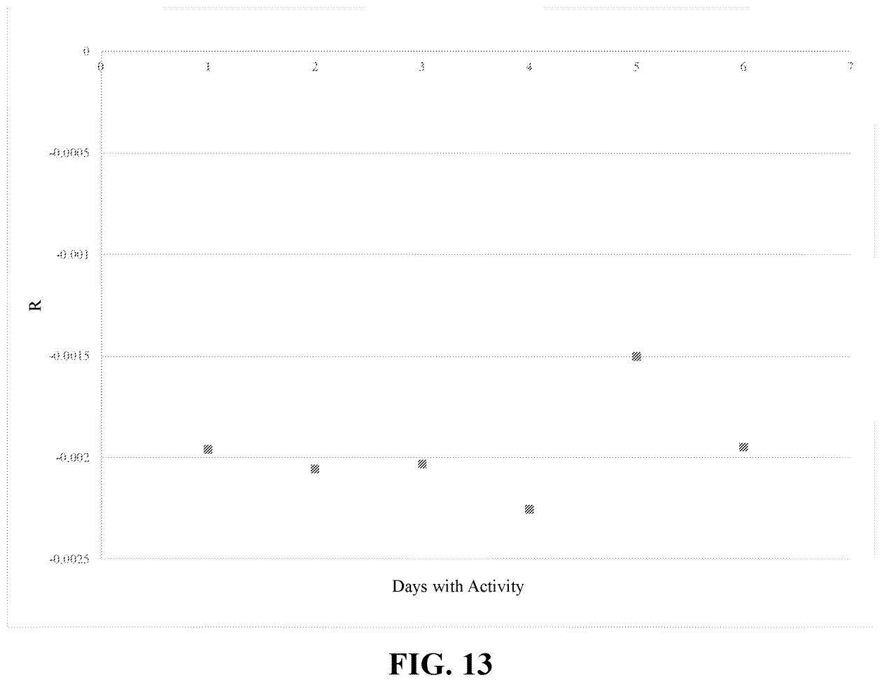

FIG. 13 shows the R values plotted against the days with activity.

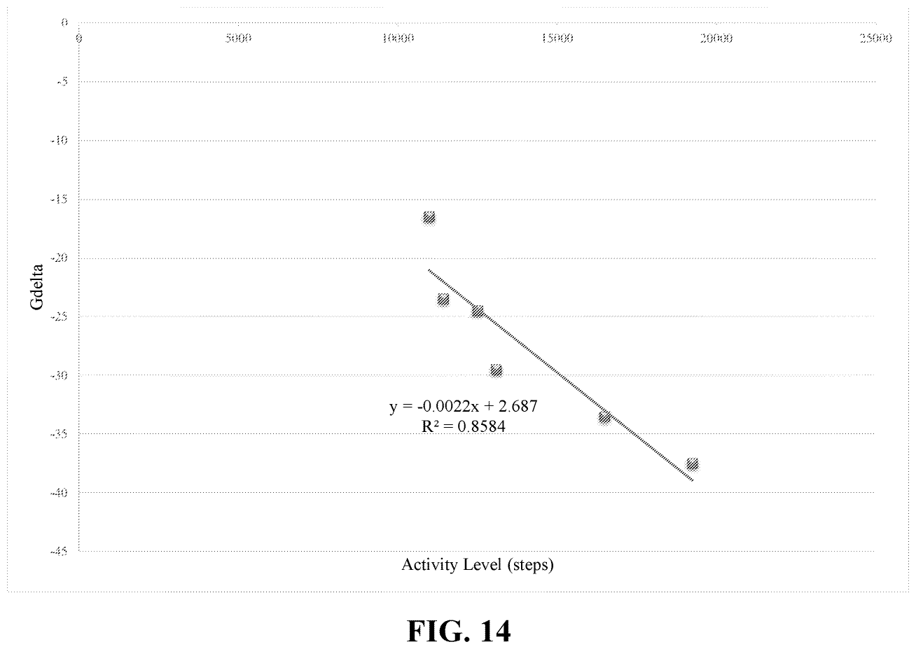

Alternatively, the median or mean of the R values can be used to represent the glycemic pattern. Further, the delta median glucose (Gdelta(Xday)) can be plotted against the activity metric (Act(Xday)) and a line fit analysis performed, resulting in the plot shown in FIG. 14.

From the line fit analysis shown in FIG. 14, the correlation coefficient R² is approximately 0.86, with an offset of 2.687 for the line fit, and a slope of −0.0022. With the analysis shown in FIG. 14, a user who wishes to engage in an activity that includes 15,000 steps, can ascertain from FIG. 14 that such activity will result in a glucose level reduction of approximately 30 mg/dL. Alternatively, the App includes a routine that estimates the upcoming overnight Gdelta(Xday) by inputting the day's activity into the linear equation. The user can then decide to take appropriate action (consume additional food/drink during or pre-activity) to better control the anticipated glucose level drop resulting from the activity.

In an alternate embodiment, the activity metric (Act(Xday)) can be categorized into two values: significant activity or not significant activity. In such a case, an overnight glucose median is associated with either a day of significant activity or with a day without significant activity, where significant activity is determined if the activity measure exceeds a predefined threshold (for example, greater than 10,000 steps for a day time period). The median day-to-night changes in median glucose level (Gd2n(Xday)) for all overnight periods associated with days with significant activity are determined, as well as the median day-to-night changes in median glucose level (Gd2n(Xday)) for all overnight periods associated with non-significant activity, and the decrease in median activity is then determined. Data sufficiency, in certain embodiments, are determined using statistical techniques; for example, by verifying that the standard error of each median calculation is below a predefined threshold, such as 20 mg/dL.

For example, the median day-to-night changes in median glucose level (Gd2n(Xday)) for all overnight periods associated with days with significant activity is determined as −28.5 mg/dL (taking the median of day-to-night changes in median glucose level for days 1, 4, 5, 6, 9, and 13—which are −26, −25, −35, −31, −18, and −39, respectively), while the median day-to-night changes in median glucose level (Gd2n(Xday)) for all overnight periods associated with non-significant activity is determined as −1.5 mg/dL (taking the median of the day-to-night changes in median glucose level for days 2, 3, 7, 8, 10, 11, 12, and 14-which are −14, 8, 17, 6, 1, −22, −11, and −4, respectively). From this, the median decrease in glucose level can be determined as −27 mg/dl (subtracting −1.5 mg/dl from −28.5 mg/dL).

In this case, the analysis result is displayed by the App to the user when subsequent significant activity is detected as follows: “For days with significant activity, glucose levels tend to be 27 mg/dL lower than for days without significant activity.” Within the scope of the present disclosure, the analysis can be expanded to any level of quantization such as three or four levels.

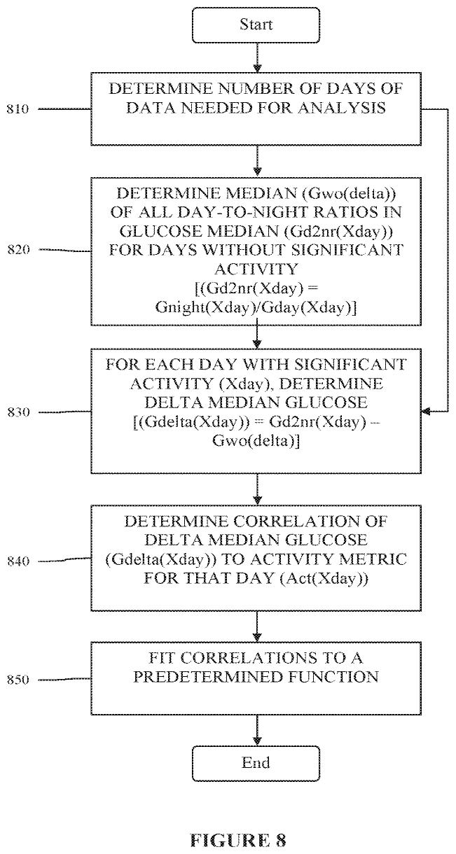

FIG. 8 is a flowchart illustrating glucose response pattern identification and characterization for a particular activity based on day-to-night glucose level ratio in accordance with one embodiment of the present disclosure. Referring to FIG. 8, the difference between the routine executed by glucose response training unit 112 of analysis module 110B (FIG. 2A) in conjunction with FIG. 7 compared to the routine shown in FIG. 8 is that instead of using the median (Gwo(delta)) of all day-to-night changes in glucose median level (Gd2n(Xday)) for days without significant activity (at step 720 in FIG. 7, the routine in FIG. 8 determines median (Gwod2nr) of all day-to-night ratios in glucose median level (Gd2nr(Xday)) for days without significant activity (820) after the number of days of data needed for analysis is determined (810). In certain embodiments, the day-to-night ratios in glucose median level (Gd2nr(Xday)) for days without significant activity is determined by dividing the median glucose level over a second predetermined time-of-day period (e.g., from 10 μm to 6 am) (Gnight(Xday)) by median glucose level over a first predetermined time-of-day period (e.g., from 8 am to 10 pm) (Gday(Xday)). That is:

$$( G d 2 nr ( Xday ) ) = Gnight ( Xday ) / Gday ( Xday )$$

Referring back to FIG. 8, the median (Gwo(delta)) of all day-to-night ratios in glucose median level (Gd2nr(Xday)) for days without significant activity is determined. The glucose response training unit 112 of analysis module 110B then determines, for each day with significant activity, the delta median glucose (Gdelta(Xday)) by subtracting each of the day-to-night ratios (Gd2nr(Xday)) for each day with significant activity (830) by the median (Gwo(delta)) of all day-to-night ratios in glucose median level for days without significant activity. In certain embodiments, after determining the number of days of data needed for analysis (810), the median (Gwo(delta)) of all day-to-night ratios in glucose median (Gd2nr(Xday)) for days without significant activity (820), and the delta median glucose (Gdelta(Xday)) for each day with significant activity (830) are simultaneously determined rather than sequentially.

Referring again to FIG. 8, similar to FIG. 7 step 740, the correlation relationship between the delta median glucose (Gdelta(Xday)) and activity metric (Act (Xday)) for each day is determined (840). This correlation relationship indicates the proportional decrease in the ratio of day-to-night glucose levels overnight after significant activity. The correlation of delta median glucose (Gdelta(Xday)) to activity metric (Act(Xday)) for the days with significant activity are fit to a predetermined function (850), and the resulting correlation information output to the user.

Referring again to the data set shown in Table 1 above, the analysis described in conjunction with FIG. 8 results in median of all day-to-night ratios in glucose median level (Gwod2nr) as 0.989991680125287, based on the median of the day-tonight ratio in glucose median level of days without significant activity as shown in Table 5 below:

TABLE 5

| | | | | | | Median of all |
|---|---|---|---|---|---|---|
| | | | Daytime | Overnight | | day-to-night ratios |
| | | Activity | Median | Median | day-to-night | in glucose median |
| | | Metric | Glucose | Glucose | ratios in glucose | without significant |
| Day | Activity? | (steps) | (mg/dL) | (mg/dL) | median Gd2nr | activity Gwod2nr |
| 2 | no | 3043 | 156 | 142 | 0.91 | |
| 3 | no | 2043 | 142 | 150 | 1.056 | |
| 7 | no | 1044 | 143 | 160 | 1.119 | |
| 8 | no | 1453 | 145 | 151 | 1.041 | |
| 10 | no | 2354 | 139 | 140 | 1.007 | |
| 11 | no | 2356 | 161 | 139 | 0.863 | |
| 12 | no | 1234 | 155 | 144 | 0.929 | |
| 14 | no | 7034 | 147 | 143 | 0.973 | 0.98999168 |

Then, the ratio of median level glucose (Gactd2nr(Xday)) for each day with significant activity can be determined by dividing the median of each day-to-night ratios in glucose median level (Gwod2nr) of 0.989991680125287 from the day-to-night ratios in glucose median (Gactd2nr(Xday)) for each day with significant activity as shown below in Table 6.

TABLE 6

| | | | | | Day-to-night | |
|---|---|---|---|---|---|---|
| | | | Daytime | Overnight | ratios in glucose | |
| | | Activity | Median | Median | median with | |
| | | Metric | Glucose | Glucose | significant | Ratio of median |
| Day | Activity? | (steps) | (mg/dL) | (mg/dL) | activity Gd2nr | glucose Gactd2nr |
| 1 | yes | 12503 | 143 | 117 | 0.818 | 0.82645323 |
| 4 | yes | 11432 | 150 | 125 | 0.833 | 0.84175792 |
| 5 | yes | 16490 | 146 | 111 | 0.76 | 0.76795996 |
| 6 | yes | 13083 | 151 | 120 | 0.795 | 0.80273603 |
| 9 | yes | 10984 | 149 | 131 | 0.879 | 0.88808285 |
| 13 | yes | 19245 | 144 | 105 | 0.729 | 0.73653818 |

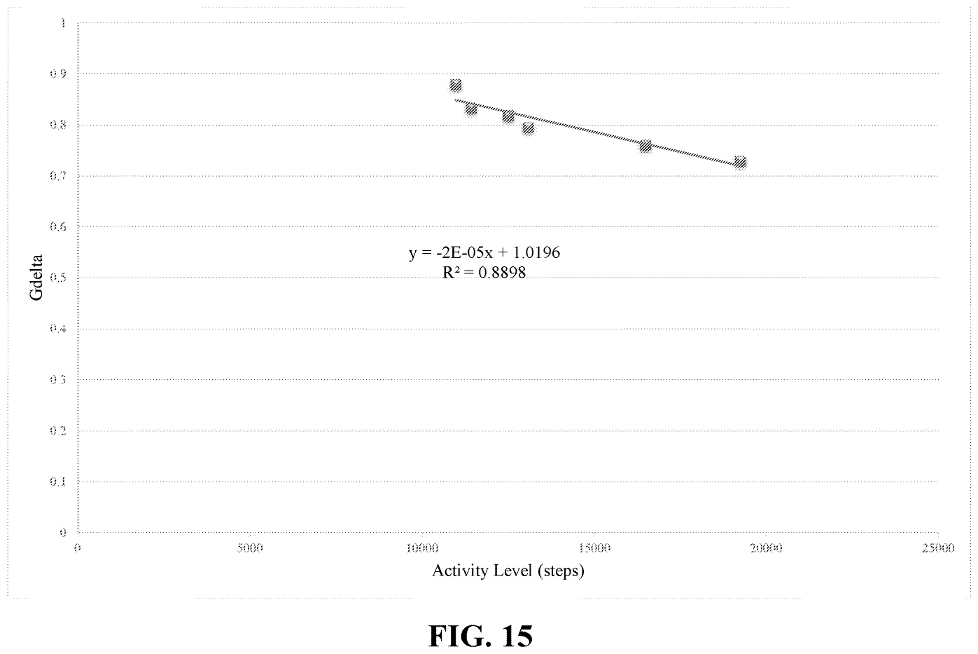

From Table 6, the median of the median glucose ratios (Gactd2nr(Xday)) for days with significant activity can be determined as 0.814595. Alternatively, a line fit analysis can be performed by plotting the median glucose ratio (Gactd2nr(Xday)) against the activity metric (Act) for days with significant activity as shown in FIG. 15.

It can be seen that the correlation coefficient R² from FIG. 15 is approximately 0.89, with an offset of approximately 1.03 and a slope of −0.00002(2E-05).

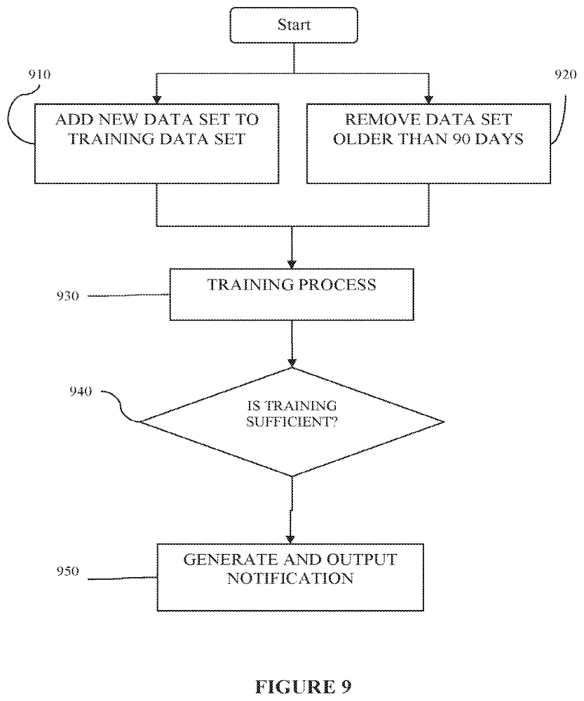

FIG. 9 illustrates a process flow for training and notification in accordance with one embodiment of the present disclosure. Referring to FIG. 9, in certain embodiments, data analysis training for example, described in conjunction with FIGS. 4-8 above, are performed on input data set received (910), at a predetermined time interval such as once daily. Every time the routine is executed, new data set that has been acquired is added to the data set maintained and used for data analysis training, for example, to determine the correlation relationship between activity and future glucose levels (e.g., overnight glucose level).

Referring back to FIG. 9, in addition to adding new data set to the training data set (910), each time the data analysis training routine is executed, older data is removed from the training set, such as data that is 90 days or older or 180 days or older or any other suitable time periods (920). This allows the data analysis training routine to adapt to the changing physiology of the user from whom the data set is derived (“forgetting”). In certain embodiments, the “forgetting” subroutine may be excluded or optional. When the data analysis training process has concluded (930), training sufficiency is checked (940) as described above in conjunction with FIG. 48 such that, for example, the uncertainty metric associated with the “fit” of the correlation relationship is less than a predetermined threshold. If it is determined that that training is sufficient (940), then notification of the results is generated and output (950). However, if it is determined that the training was insufficient, then no notification is generated or output. Alternatively, in certain embodiments, rather than providing no notification when the App determines that the training was insufficient, a notification indicating that training is not yet sufficient may be provided.

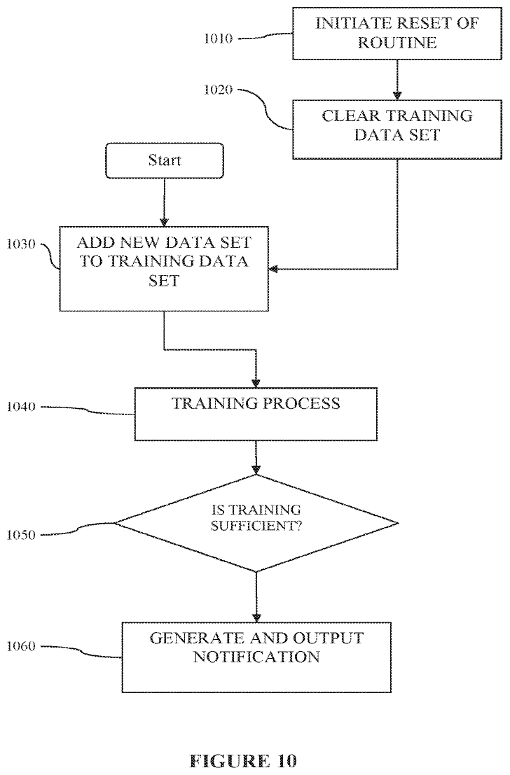

FIG. 10 illustrates a process flow for training and notification in accordance with another embodiment of the present disclosure. As shown in FIG. 10, the data analysis training and notification routine is similar to the routine shown and described in FIG. 9, with the “forgetting” feature (920) replaced by a reset or clearing the training data set (1010 and 1020). Referring to FIG. 10, the initiating reset of routine (1010) and clearing the training data set (1020) in certain embodiments are implemented in response to actuation of an input button for example, on the user interface of the App to reset the training routine. In certain embodiments, the user initiates the reset of the routine (1010) and the training data set clears (1020) so as to update the learned correlation relationship between activity and future glucose levels by the App.

Referring to FIG. 10, when the reset is initiated, then the data training and notification routine is invoked periodically thereafter, and similar to the routine shown in FIG. 9, the new data set is added to the training data set (1030) and after the training process is complete (1040), it is determined whether the training is sufficient (1050). When it is determined that the training is sufficient, the App in certain embodiments generates and outputs notification to the user (1060). When it is determined that the training was insufficient (1060), then no notification is presented to the user, or alternatively, a notification indicating that the training was insufficient is generated by the App and presented to the user.

Within the scope of the present disclosure modifications to the data set training and notification routines described in conjunction with FIGS. 9 and 10 are contemplated where both the reset/clearing training data set (1010-1020, FIG. 10) feature and the “forgetting” feature (920, FIG. 9) are included in the same analysis routine. Also, in certain embodiments, the reset occurs periodically, such as once per year. Alternatively, in certain embodiments, the reset occur after the training has provided a valid notification (i.e., when it is determined that the training was sufficient).

In the manner described, in accordance with the embodiments of the present disclosure, Type-1 diabetic patients, Type-2 diabetic patients as well as pre-diabetics are provided with tools to monitor physiological conditions while engaged in daily routines and over time the App, for example, executable on a mobile phone of the user or the patient provides consistent glucose response to various types of activities and parameters that may impact the fluctuation in the user or the patient's glucose level. Such tools will allow the user or the patient to modify diet, exercise routine, or other daily activities knowing how the particular diet, exercise or activity affects the fluctuation in glucose level, and proactively take action to maintain the desired glycemic control and avoiding harmful glycemic excursions.

Embodiments of the present disclosure include aspects of data collection including detecting a particular activity and prompting the user or the patient to enter additional information related to the detected activity so as to render the data collection more robust. For example, using the activity monitor 130A, when the App executed on the mobile phone 110 detects continuous movement for a predetermined time period, the App, in certain embodiments, is configured to generate and output a query to the user interface 110A to prompt the user or the patient to either confirm that the detected activity is occurring, and/or add additional information related to the detected activity (which prompts, in certain embodiments, may be generated and output to the user interface 110A upon detection of the termination of the activity).

In this manner, in accordance with the embodiments of the present disclosure, robust physiological parameter monitoring system and dynamic glucose response pattern to provide consistent and reliable glucose response to physiological or other parameters and activities is provided.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

1. A system for predicting an overnight glucose fluctuation for a user, comprising:
one or more data analysis modules configured to:
receive bolus insulin information and glucose level information over a predetermined time period;
determine an overnight glucose response pattern for the user based on the received glucose level information and the received bolus insulin information;
receive current glucose level information of the user for a current time period;
dynamically update the glucose response pattern based on the current glucose level information; and
predict, based on the updated glucose response pattern, whether an overnight glucose fluctuation exceeds a predetermined range; and
a data output interface in communication with the data analysis module to output a notification associated with the predicted overnight glucose fluctuation.

2. The system of claim 1, wherein the one or more data analysis modules is further configured to update the overnight glucose response pattern based on a received amount of medication to be consumed.

3. The system of claim 1, wherein the one or more data analysis modules is further configured to update the overnight glucose response pattern based on a received amount of food to be consumed.

4. The system of claim 1, wherein the one or more data analysis modules is further configured to update the overnight glucose response pattern based on a received activity metric information over the predetermined time period.

5. The system of claim 4, wherein the activity metric information includes an amount of calories burned.

6. The system of claim 4, wherein the activity metric information includes a number of steps recorded.

7. The system of claim 4, wherein the activity metric information includes one or more of a time duration of an activity, an intensity level of an activity, heart rate data associated with an activity, a location of an activity including altitude, a distance traveled during an activity, or a type of an activity.

8. The system of claim 1, wherein the notification includes one or more of a visual, auditory, or vibratory notification.

9. The system of claim 1, wherein the one or more data analysis modules is further configured to output the updated glucose response pattern.

10. The system of claim 1, wherein the predetermined time period includes a plurality of days.

11. The system of claim 1, wherein the notification comprises a therapy recommendation.

12. The system of claim 11, wherein the therapy recommendation includes a recommendation to consume one or more of a medication, food, or drink.

13. The system of claim of 1, further comprising a glucose sensor configured to sense a glucose level in an interstitial fluid of a user, the glucose sensor including a working electrode comprising an analyte-responsive enzyme.

14. A method for predicting an overnight glucose fluctuation for a user, comprising:
determining an overnight glucose response pattern for the user based on a received glucose level information and a received bolus insulin information;
receiving current glucose level information of the user for a current time period;
dynamically update the glucose response pattern based on the current glucose level information;
predicting, based on the updated glucose response pattern, whether an overnight glucose fluctuation exceeds a predetermined range; and
outputting a notification associated with the predicted overnight glucose fluctuation.

15. The method of claim 14, further comprising updating the overnight glucose response pattern based on a received amount of medication to be consumed.

16. The method of claim 14, further comprising updating the overnight glucose response pattern based on a received amount of food to be consumed.

17. The method of claim 14, further comprising updating the overnight glucose response pattern based on a received activity metric information over the predetermined time period.

18. The method of claim 17, wherein the activity metric information includes an amount of calories burned.

19. The method of claim 17, wherein the activity metric information includes a number of steps recorded.

20. The method of claim 17, wherein the activity metric information includes one or more of a time duration of an activity, an intensity level of an activity, heart rate data associated with an activity, a location of an activity including altitude, a distance traveled during an activity, or a type of an activity.

21. The method of claim 14, wherein the notification includes one or more of a visual, auditory, or vibratory notification.

22. The method of claim 14, further outputting the updated glucose response pattern.

23. The method of claim 14, wherein the predetermined time period includes a plurality of days.

24. The method of claim 14, wherein the notification comprises a therapy recommendation.

25. The method of claim 24, wherein the therapy recommendation includes a recommendation to consume one or more of a medication, food, or drink.

26. The method of claim 14, wherein receiving glucose level information includes sensing a glucose level in an intertitial fluid of a user using a glucose sensor, the glucose sensor including a working electrode comprising an analyte-responsive enzyme.
